(19) United States
(12) Patent Application Publication
(10) Pub. No.: US 2006/0173396 A1
(43) Pub. Date: Aug. 3, 2006

(54) Systems and methods for temperature adjustment using bodily fluids as a thermic medium

(76) Inventors: Mehdi Hatamian, Coto De Caza, CA (US); Mehrtosh Ghalebi, Rancho Santa Margarita, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/297,116

(22) Filed: Dec. 8, 2005

Related U.S. Application Data
(60) Provisional application No. 60/634,922, filed on Dec. 9, 2004.

(30) Foreign Application Priority Data
Dec. 8, 2005 (WO) PCT/US05/44317

(51) Int. Cl. A61M 37/00

(52) U.S. Cl. 604/6.13; 604/6.11; 422/44

(57) ABSTRACT

In some embodiments, there is provided an apparatus for affecting the temperature of a bodily organ using a bodily fluid as a thermic medium; other embodiments provide a method of using the apparatus. The apparatus can include a fluid-drawing device such as a needle, an extra-bodily fluid pathway that can comprise a flexible medical tube, a temperature-affecting device such as a Peltier cooler or resistive heater a pump such as a peristaltic pump, and a fluid-insertion device. The temperature affecting device can be in thermal contact with the extra-bodily fluid pathway.

### RELATED APPLICATION

This application claims priority to pending U.S. Provisional Patent Application No. 60/634,922, filed Dec. 9, 2004, entitled SYSTEMS AND METHODS FOR TEMPERATURE ADJUSTMENT USING BODILY FLUIDS AS A THERMIC MEDIUM, the entirety of which is hereby incorporated by reference herein and made part of this specification.

### BACKGROUND OF THE INVENTION

1. Field of the Invention

The disclosed embodiments relate to systems and methods for controlling the temperature of a region or organ in a body using bodily fluids as a thermic medium.

2. Description of the Related Art

Under some clinical circumstances, it is desirable to increase or decrease the temperature of a specific organ or anatomical region of the body. For example, after a person has been severely wounded, it is sometimes beneficial to induce hypothermia to reduce swelling and provide other clinical benefits. One example of an anatomical region that can be cooled is a human brain.

Current methods of cooling the brain have significant undesirable consequences. For example, cooled helmets and ice-baths have been known to cause extreme discomfort, and even frostbite in subjects. Furthermore, such approaches are ineffective at cooling the brain uniformly, causing a large temperature gradient from a very cold scalp to largely un-cooled internal brain tissue. Moreover, such external techniques are incapable of reducing the temperature of the target tissue quickly enough, or in a controllable manner, for good clinical effect.

### SUMMARY

In some embodiments there is provided an apparatus for affecting the temperature of a bodily organ. Such an apparatus can comprise a fluid-drawing device, and extra-bodily fluid pathway, a temperature-affecting device in at least partial contact with the extra-bodily fluid pathway, a pump, and a fluid-insertion device. In some embodiments, there is provided an apparatus wherein the fluid-drawing device and fluid-insertion device are both needles. The temperature-affecting device can be a thermal electric device, a Peltier device, a resistive heater, and/or a heat sink, for example. The extra-bodily fluid pathway can comprise medical tubing, that can be sterilized, and/or disposable. The extra-bodily fluid pathway can be configured to increase thermal contact between the extra-bodily fluid pathway and the temperature changing device. In some embodiments, the extra-bodily fluid pathway can comprise a tube that is curved along a region of thermal contact with a portion of the temperature changing device. In some embodiments, the extra-bodily fluid pathway further comprises a first outflow portion configured to contain fluid flowing from a first living entity and a second inflow portion configured to contain fluid flowing to a second living entity. The first and second living entity can, in some embodiments, be a single human orgnanism, for example. In some embodiments, the first and second living entities can be the same living organ, or different living organs. In some embodiments, the inflow portion is shorter than the outflow portion. In some embodiments, the pump is a peristaltic pump. In some embodiments, there is further provided a pump motor. In some embodiments, a pump motor can interface with a control unit, which can also interface with the temperature-affecting device. In some embodiments, the control unit is configured to control the temperature of the temperature-affecting device and/or control the pump speed.

In some embodiments, there is provided a method of changing the temperature of a portion of a living organism comprising: providing a temperature controller, providing a fluid assembly in thermal contact with said temperature controller, introducing a fluid from the living organism into the fluid assembly, causing a fluid to flow through the fluid assembly, and subsequently reintroducing the fluid into the living organism. In some embodiments, the temperature controller can be a thermal electric device. In some embodiments, the temperature controller can be a Peltier device. In some embodiments, the temperature controller can comprise a heat exchanger. In some embodiments, the temperature controller can comprise a thermal couple. In some embodiments, having a heat exchanger, the temperature controller can comprise a coil plate. In some embodiments, providing a temperature controller can comprise providing a control fluid. In some embodiments, providing a temperature controller can further comprise providing a dipping torus. In various embodiments, providing a fluid assembly can comprise one or more of the following: providing a heat exchanger, providing a sterile medical tube, or providing a disposable fluid tight assembly. In some embodiments, introducing fluid from the living organism into the fluid assembly can comprise one or more of the following: inserting a needle into the living organism, attaching a tube to a fluid passageway that is in fluid communication with a fluid passageway in the living organism, and/or introducing blood into the fluid assembly. In some embodiments, causing fluids to flow through the fluid assembly can comprise one or more of the following: Using a peristaltic pump to urge fluid through the fluid assembly, allowing a heart to pump blood through the fluid assembly, and/or establishing a pressure gradient to cause fluid flow. In various embodiments, re-introducing the fluid into the living organism can comprise one or more of the following: inserting a needle into the living organism and/or allowing fluids to flow from the fluid assembly through an exiting passageway into a fluid passageway inside the living organism. In some embodiments, there is provided a method that further comprises the measuring and controlling of the temperature of the fluid. In some embodiments, measuring and controlling the temperature of the fluid can comprise using a computer. In some embodiments, there is provided a method-that further comprises measuring and controlling the flow rate of the fluid, which can also be done using a computer for example. In some embodiments, a further step comprises allowing bodily fluids to flow through a portion of a living organism that has been severed from another portion of a living organism. In some embodiments, there is provided a further aspect comprising and detaching the fluid assembly from the temperature controller. In some embodiments, the fluid assembly can be attached to a second temperature controller.

In some embodiments, the method further comprises improving thermal conduction using a thermally-conductive substance. In some embodiments, the method further comprises attaching or connecting multiple temperature controllers in a series. In some embodiments, the method further comprises attaching or connecting multiple temperature controllers in parallel.

In some embodiments, there is provided a system for controlling organ temperature. The system can include an extra-corporeal fluid path adapted to be connected to a living organ; a temperature controller; a pump; and a fluid that follows a fluid path having a first portion within the living organ and a second portion within the temperature controller. In some embodiments, the extra-corporeal fluid path is adapted to be connected to a brain. In some embodiments, the temperature controller is a heating mechanism. In some embodiments, the temperature controller is a cooling mechanism. In some embodiments, the temperature controller is a thermoelectric device. In some embodiments, the temperature controller is a Peltier device. In some embodiments, the pump is a living heart, whereas in some embodiments, the pump is an artificial heart. In some embodiments, the pump is a peristaltic pump. In some embodiments, the fluid is blood. In some embodiments, the fluid is lymphatic fluid. In some embodiments, the fluid is interstitial fluid. In some embodiments, the portion of the fluid path within the temperature controller is not within the living organ. In some embodiments, the portion of the fluid path within the living organ comprises blood vessels. In some embodiments, the portion of the fluid path within the temperature controller comprises a disposable portion. In some embodiments, the system further comprises a computer.

### BRIEF DESCRIPTION OF THE DRAWINGS

FIG. 1 depicts a system in accordance with embodiments of the disclosed inventions.

FIG. 2 illustrates an embodiment of a system for heating or cooling bodily fluids.

FIG. 3 depicts another view of the system of FIG. 2.

FIG. 4 illustrates an embodiment of an electrical assembly.

FIG. 5 depicts another view of the embodiment of FIG. 4.

FIG. 6 depicts another view of the embodiment of FIG. 4.

FIG. 7 depicts a line drawing of the embodiment of FIG. 4.

FIG. 8 illustrates an embodiment of a motor casing.

FIG. 9 illustrates an embodiment of a motor.

FIG. 10 illustrates an embodiment of a motor bracket.

FIG. 11 illustrates an embodiment of an electrical assembly housing.

FIG. 12 illustrates an embodiment of a heat sink.

FIG. 13 depicts a schematic representation of a Peltier module.

FIG. 14 illustrates an embodiment of cold plate.

FIG. 15 illustrates an embodiment of a bracket.

FIG. 16 illustrates how a thermoelectric system can function.

FIG. 17 illustrates an embodiment of a fluid assembly housing.

FIG. 18 illustrates embodiments of coil plates and a fluid assembly housing.

FIG. 19 depicts another view of the embodiment of FIG. 17.

FIG. 20 illustrates an embodiment of a top coil plate.

FIG. 21 illustrates an embodiment of a bottom coil plate.

FIG. 22 depicts a fluid assembly.

FIG. 23 illustrates a system in accordance with disclosed embodiments.

FIG. 24 illustrates a different view of the system of FIG. 23.

FIG. 25 illustrates a different view of the system of FIG. 23.

FIG. 26 illustrates a different view of the system of FIG. 23.

### DETAILED DESCRIPTION OF THE PREFERRED EMBODIMENTS

Fluid that naturally circulates through the body can be used as a thermic medium for heating or cooling the body, or portions of the body. The fluid channels passing through various parts of the body thoroughly penetrate the various organs, body tissues and systems. This penetration can be utilized to good effect for adjusting body temperature. Properties of fluids such as specific heat and flow capabilities can provide advantages to such a temperature control system. Such temperature adjustment can be accomplished on any living entity that has internal fluidic passages.

Mammals are examples of living entities for which such a system can be effectively used. For example, mammal brains can be cooled to induce certain results. Blood can be used as the thermic medium, and a human brain can be targeted for cooling. Cooling a mammal's brain can be an effective way of treating trauma. For example, cooling a subject's brain and the blood flow to the brain can slow internal hemorrhaging, induce a beneficial reversible coma, and/or advantageously slow various metabolic processes. Such a cooling process can be beneficial to humans under certain circumstances. Hypothermia can be induced in a controlled way, potentially limited to specific regions of the human's body. For example, when the brain is cooled slightly, brain hypothermia can be induced and a temporary sleep-like state can result. Such induced hypothermia can reduce the brain's oxygen demand and minimize swelling.

There can also be benefits to raising the temperature of a particular organ or region of the body. A human who has been exposed to extreme temperatures can be treated by controlling the temperature of the human's bodily fluids. In particular, a human's body temperature can be gradually and beneficially raised by warming the human's blood. For example, the temperature of the entire body can be raised to combat potential hypothermia that may occur during open surgery. Hyperthermia can be induced in a controlled way, potentially limited to specific regions of the human's body.

This specification discloses devices and systems that can be used to advantageously adjust fluid temperatures, as well as methods of controlling fluid temperatures. This information can be used to great advantage by humans in a medical setting, for example. This information can also be used to great advantage to treat wounded soldiers on the field of battle, for example.

Some embodiments can improve upon current methods of heating or cooling tissue in a target region by using bodily fluid as a thermic medium. Blood can be removed, for example, and reintroduced into a body after having been cooled or heated. Indeed, an apparatus for affecting the temperature of a bodily organ such as described herein has many benefits.

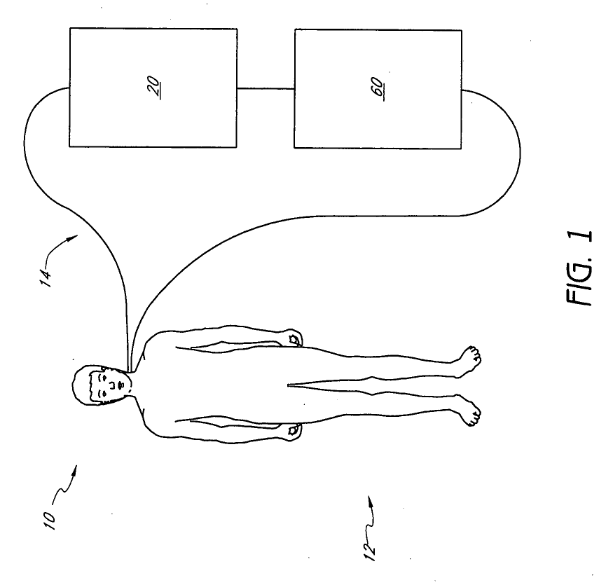

In some embodiments, a thermic system 10 comprises a temperature controller 20 and a pump 60. The system 10 can further comprise a fluid path 14. Fluid can be drawn from the patient 12 at a desired region of the body using a fluid-drawing device such as a needle, needle-less valve, catheter, etc. Blood can be drawn from an appendage such as a leg, for example. Blood can also be drawn from near a wound, for example. FIG. 1 illustrates a fluid path 14 originating and terminating in the region of a patient's neck.

Once drawn from the body, the fluid can pass through fluid path 14 to temperature controller 20, where the fluid is affected or adjusted (e.g., heated or cooled) to a temperature above or below its normal temperature. The fluid can then pass through a continuation of fluid path 14 to pump 60, which urges the fluid to flow through the system. The fluid can then continue through fluid path 14 and return to the body of the patient 12. In some embodiments, the pump 60 can precede the temperature controller 20 in the fluid path 14. In some embodiments, the heart can perform the pumping function and a separate pump such as pump 60 may not be necessary.

The system 10 can comprise various components (not shown) that have been developed for use with dialysis systems to minimize some of the potential adverse effects of removing bodily fluid and reintroducing the fluid into a body. For example, such components can help combat electrolyte depletion, clot development, and plaque deposition. Components can also be included to help create a gradual flow. Such components can include, for example, one or multiple control units that interface(s) with each of the other components to control various parameters and provide feedback to a user.

The temperature controller 20 can comprise a cooling system. The cooling system can capitalize on a state change of matter to create a cooling effect, using a fan to create air flow over water for a simple evaporative cooling effect, for example. The cooling system can be a conventional refrigerator and comprise a pressurized refrigerant, a compressor, an evaporator, and a condenser. The cooling system can comprise a thermoelectric cooler, such as a Peltier cooler, for example. In some embodiments, the cooling system can take advantage of the Peltier-Seebeck effect. In certain embodiments, the temperature controller 20 can comprise an electrical power source.

The temperature controller 20 can comprise a heating system. The heating system can utilize a variety of known heating mechanisms, including, without limitation, electrical or resistive heating, combustion heating, and/or electromagnetic heating using LASER, microwave, or solar energy, for example. The temperature controller can also combine cooling and heating capabilities.

The pump 60 can comprise any fluid pump. One advantageous embodiment employs a peristaltic pump that urges fluid through the system without any need for valves. This can allow the fluid to remain isolated in generally sterile environment inside a tube, for example. The pump 60 can comprise a power source that provides electrical energy for running the system. For example, any source of alternating or direct current may be used, such as a battery, a car battery, or a municipal power source accessed through a wall outlet. In some embodiments, a system 10 can use multiple types of power sources.

Fluid path 14 can be through any fluid passage, tube, or pathway. For example, ANSI standard medical tubing of various widths can be used. One specific example is TYGON® tubing. Blood, for example, can flow from the patient's arteries or veins into the tubing through medical needles. The tubing diameter can be chosen to provide a desired fluid flow rate. Furthermore, the length of the fluid path 14 can be adjusted according to various parameters. Advantageous embodiments provide a short fluid path after the fluid exits the fluid control system and before the fluid reenters the subject. This can minimize unwanted temperature change of the fluid. Advantageous embodiments provide for a shorter overall length of fluid path 14 to minimize the amount of fluid required to fill the system. This can minimize adverse health consequences of removing too much blood from the body, such as brain stem collapse, organ atrophy, tissue necrosis, organ failure, oxygen debt, and shock, for example. A short fluid path 14 can also allow for lower flow rates, minimizing the volume of blood outside the body. The fluid path 14 can be advantageously configured to maximize the path length inside a temperature control device, while minimizing the path length between the device and the body. This configuration can provide higher portability and system efficiency, for example.

In some embodiments, the fluid pathway is shorter than approximately 50 inches. In some embodiments, the fluid pathway is shorter than approximately 30 inches. In some embodiments, the fluid pathway is shorter than approximately 20 inches. In a preferred embodiment, the fluid pathway is approximately 12 inches long.

After the temperature of the bodily fluid has been affected or adjusted, the fluid can be returned to the target region. With blood drawn from and returned to one or multiple carotid arteries, for example, the blood flow to the brain can be cooled. If the blood is returned to a single carotid, the corresponding region of the brain can be cooled. In some embodiments, the whole brain can be cooled through cross-circulation, even when the cooled blood is returned to a carotid artery on one side of the body. Alternatively, the blood may be returned to carotid arteries on each side of the body, for example.

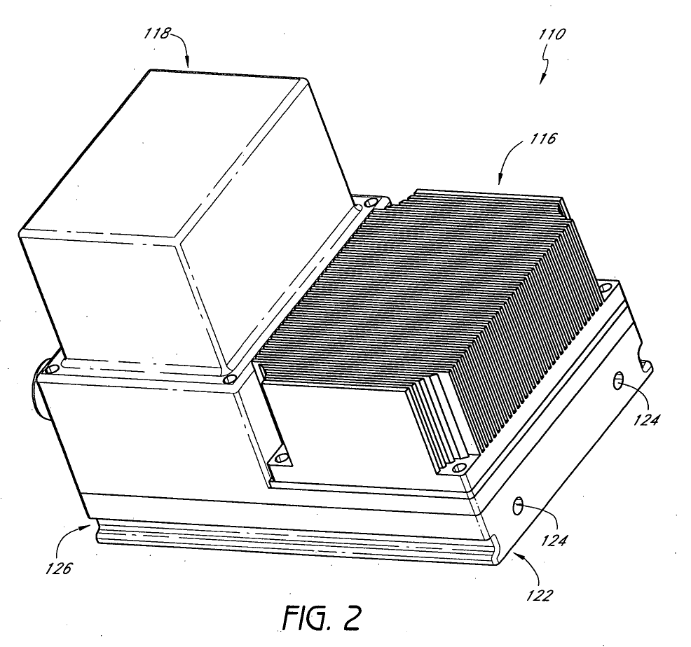

FIG. 2 illustrates an example of the system 110 for heating or cooling bodily fluids such as blood, lymphatic fluid, or interstitial fluid, for example. The system 110 can comprise a heat sink 116, a motor casing 118, and a fluid assembly 122, as illustrated. The fluid assembly can comprise two holes 124 that can be used for tubing having a fluid inside. As the illustration depicts, heat sink 116 can comprise multiple metal plates arranged in a generally aligned and closely-spaced configuration. The system 110 can also comprise a groove 126 that extends along the entire length of the fluid assembly 122 as shown. The groove can be used to mechanically attach the system 110 to another medical device or a portability component (not shown), for example. The system 110 can be used in combination with other similar systems. For example, if fluid flows through one such system 110 and subsequently flows through another such system in series with system 110, an overall cooling or heating effect can be increased. If fluid flows through a system comprising multiple systems such as system 110 that are arranged in parallel, for example, an overall capacity to control temperature for greater volumes of fluid simultaneously can be achieved.

Advantageously, system 110 can be configured to lower the temperature of an organ such as the brain by 3 degrees. The temperature parameters of such a system advantageously account for cooling and heating that occurs inside the body from passage of fluid through muscle tissue, which can provide a large source of heat in a body. Because the brain contains relatively little muscle tissue, such a heating effect is lessened in the brain. A set point can be chosen for the operating temperature of the device. The set point can become a clinically-determined default for portable units, or it can be determined by a person according to appropriate medical information available to the user.

System 110 is advantageously configured to be portable. For example, the system can be attached to an injured patient and hung around the patient's neck on a cord, placed in a pocket of the patient's clothing, or otherwise carried with the patient during transportation to a hospital, for example. Furthermore, system 110 can be configured to be light, easily manufactured, and inexpensive. Such a device can be used as a standard supply in trauma units and in the supplies carried by medics and in ambulances. Moreover, the system 110 can be compatible with a non-portable hospital system. For example, fluid assembly 122 can be removed from electrical assembly 134 while fluid assembly 122 is still attached to a patient. Thus, when a patient arrives at a hospital, the same needles, tubing, and fluid assembly can be used and a more complex hospital system for controlling blood temperature can be employed in place of electrical assembly 134. Advantageously, a portion of the system such as fluid assembly 122 can be disposable, thus reducing the cost of the device and allowing for sanitized replacement portions and a compatible reusable portion.

In some embodiments, a length (measured in the same direction as the elongated direction of the groove 126) of a system such as system 110 can be less than approximately 30 inches. In other embodiments, the length can be less than 20 inches. In other embodiments, the length can be less than 10 inches. In a preferred embodiment, a length of the system 110 is approximately 4.7 inches.

In some embodiments, a height of a system such as system 110 can be less than approximately 30 inches. In other embodiments, the height can be less than 20 inches. In other embodiments, the height can be less than 10 inches. In a preferred embodiment, a height of the system 110 is approximately 3.7 inches.

In some embodiments, a width of a system such as system 110 can be less than approximately 30 inches. In other embodiments, the width can be less than 20 inches. In other embodiments, the width can be less than 10 inches. In a preferred embodiment, a width of the system 110 is approximately 3.1 inches.

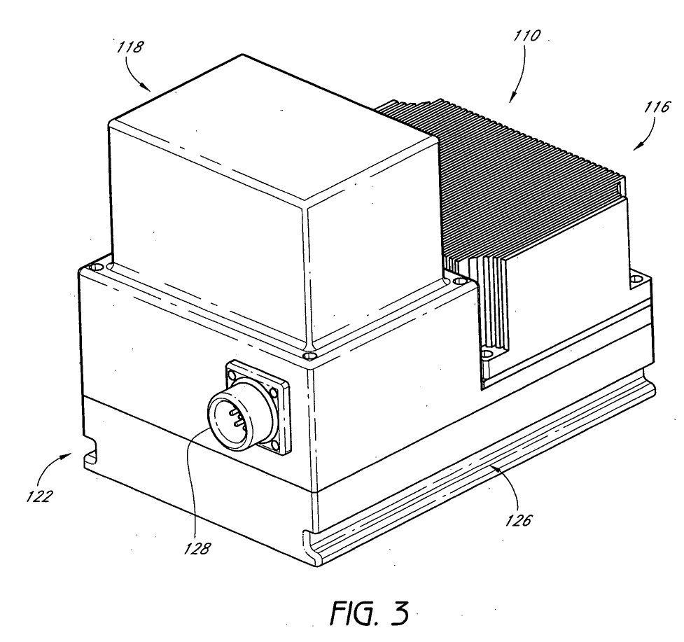

FIG. 3 illustrates another view of the same embodiment depicted in FIG. 2. This view also illustrates a motor casing 118, a heat sink 116, and a groove 126. In addition, a plug receiver 128 is shown in FIG. 3. Plug receiver 128 can lead to an external power source such as a vehicle battery or wall outlet. The power source can supply electrical power to both the motor and the Peltier cooling device. Plug receiver 128 can also connect the device to a control unit (not shown). The control unit can allow the user to control the speed and direction of the motor 142, the direction and amount of voltage or current provided by the power supply to the thermoelectric module (such as Peltier module 164, discussed below), and any other temperatures, flow rates, or controllable components. The control unit can supply electrical current to the thermoelectric module in either direction, allowing the module to heat or cool the fluid as needed. The control unit can be configured to detach from system 110 even while system 110 is operating. Thus, the control unit can be a larger, less portable unit than the system 110.

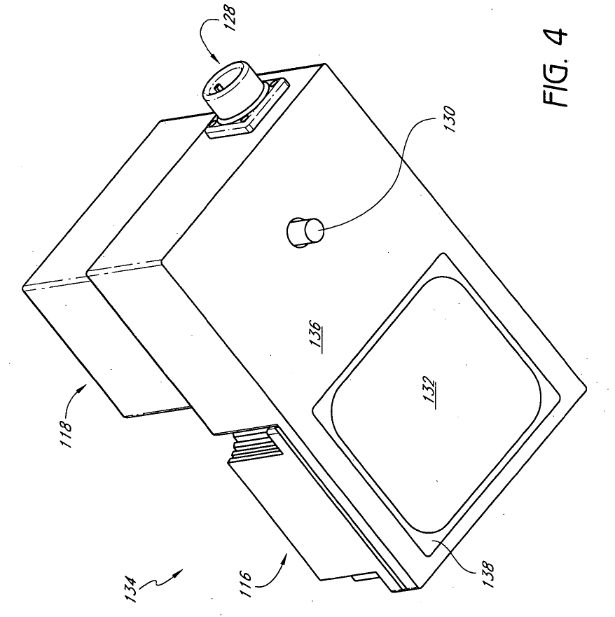

FIG. 4 depicts electrical assembly 134, a part of the system 110. Fluid assembly 122 has been separated from electrical assembly 134. Electrical assembly 134 comprises motor casing 118, heat sink 116, cold plate 132, motor spindle 130, and plug receiver 128. The bottom surface 136 of electrical assembly 134 is flush with the surface of cold plate 132. In contrast, motor spindle 130 protrudes beyond bottom surface 136, and is configured to extend into a portion of fluid assembly 122. Bracket 138 holds cold plate 132 in place in bottom surface 136. The surface of bracket 138 is flush with cold plate 132 and with bottom surface 136.

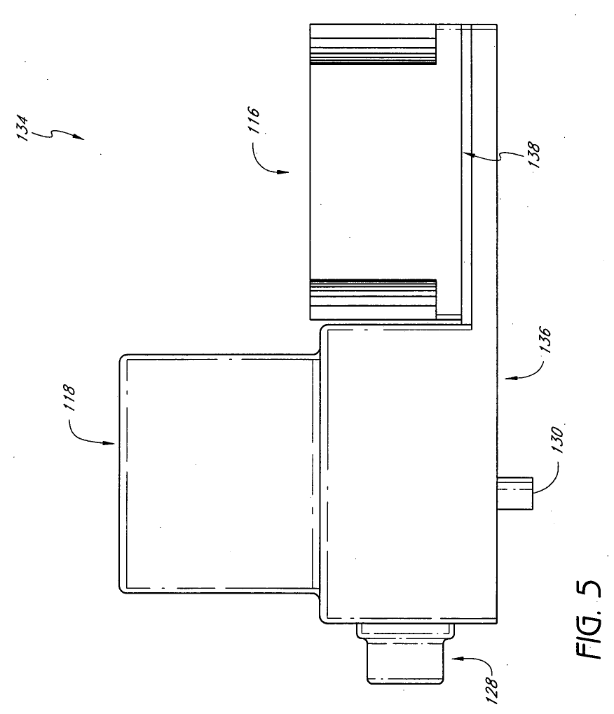

FIG. 5 illustrates another view of electrical assembly 134. Protruding from the top of electrical assembly 134 is motor casing 118. Protruding to the left in this figure is plug receiver 128. Extending below surface 136 is motor spindle 130. Heat sink 116 is also visible. Below heat sink 116 can be found bracket 138 for holding a thermoelectric module (not shown). Heat sink 116 is positioned on the Peltier module 164 (discussed below)—which is positioned inside bracket 138. Heat sink 116 and the Peltier module 164 can be in direct thermal contact.

In some embodiments, a plate comprising part of a heat sink has long dimensions of less than 10 inches by less than 10 inches. In a preferred embodiment, the dimensions can be less than 5 inches by less than 5 inches. In a more preferred embodiment, the dimensions can be approximately 2.3 inches by approximately 1.2 inches. In a preferred embodiment, the thickness of each plate is much less than either of these dimensions.

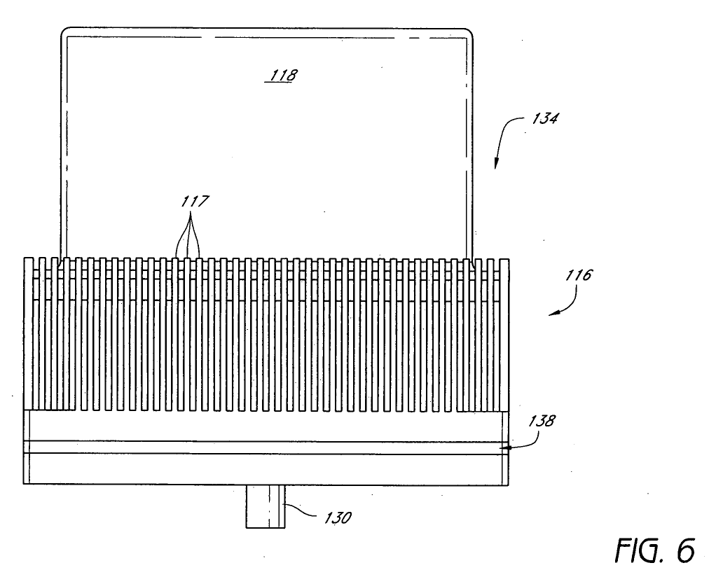

FIG. 6 illustrates another view of electrical assembly 134. Motor casing 118 is shown in this view. Motor spindle 130 can also be seen at the bottom of the figure. Heat sink 116 is shown immediately above bracket 138. This figure illustrates an end arm view of plates 117 that comprise heat sink 116. As this figure illustrates, plates 117 can be arranged in a line, with each plate directly parallel to the plane of each adjacent plate 117. In this way each plate 117 can contact the air around approximately its entire surface and the plates can still be arranged in a compact manner.

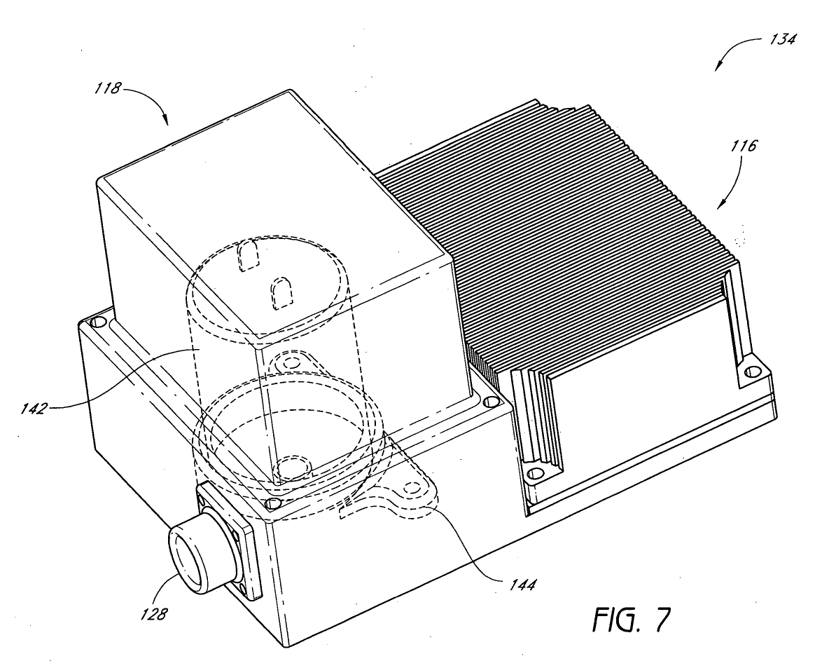

FIG. 7 is a line drawing showing some of the internal structure of electrical assembly 134. The outline of motor casing 118 is shown, as well as heat sink 116 and plug receiver 128. Inside motor casing 118 can be seen motor 142 and motor bracket 144. Motor bracket 144 has a circular opening that surrounds and holds motor 142. Motor 142 has a generally cylindrical shape, as illustrated.

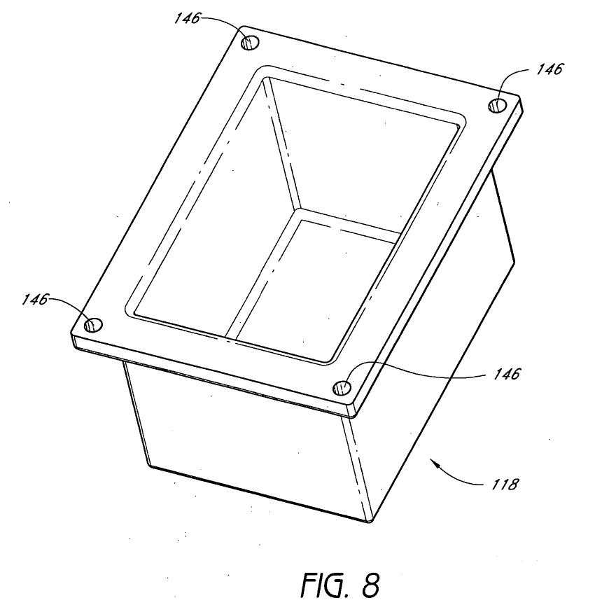

FIG. 8 illustrates the top portion of motor casing 118 separated from electrical assembly 134. From this perspective, it is clear that motor casing 118 has a hollow inside for the motor 142. Holes 146 allow the top of motor casing 118 to be fastened to electrical assembly 134 using screws for rivets, for example.

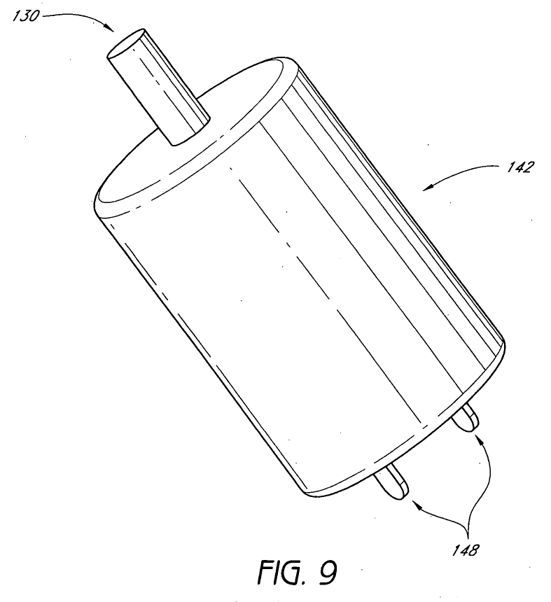

FIG. 9 illustrates an embodiment of a motor 142. The motor 142 has a motor spindle 130 protruding from one end. The motor 142 has two prongs 148 protruding from the opposite end. Various types and sizes of motors can be employed to drive the pump and provide power to the system. The motor can be powered by battery, AC current, or solar cells, for example.

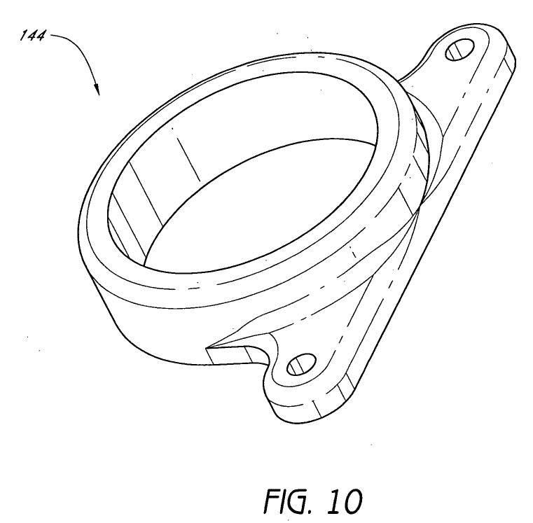

FIG. 10 depicts motor bracket 144. The motor bracket 144 can be used to position the motor 142 within motor casing 118 and to align motor spindle 130 appropriately with a fluid pump (discussed below).

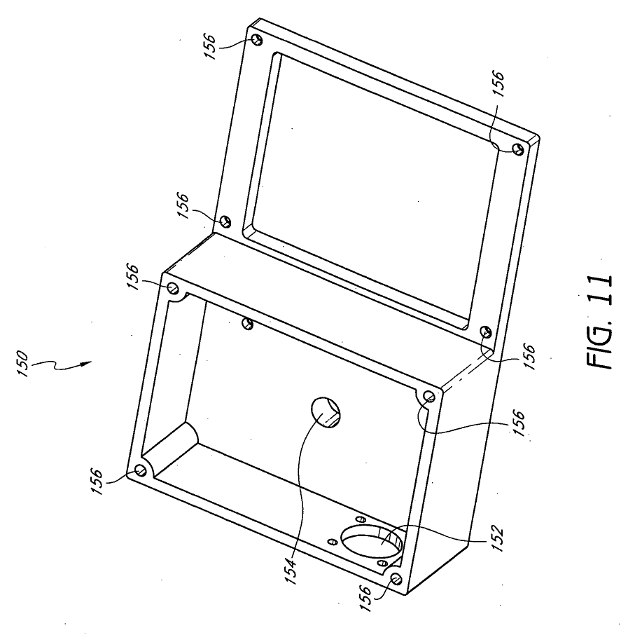

FIG. 11 shows an electrical assembly housing 150. The electrical assembly housing 150 comprises an opening 152 through which the plug receiver 128 can protrude. The housing 150 also comprises a spindle hole 154 through which the motor spindle 130 can protrude. The housing 150 also includes several screw holes 156, into which screws can be inserted to hold the other components to the housing 150.

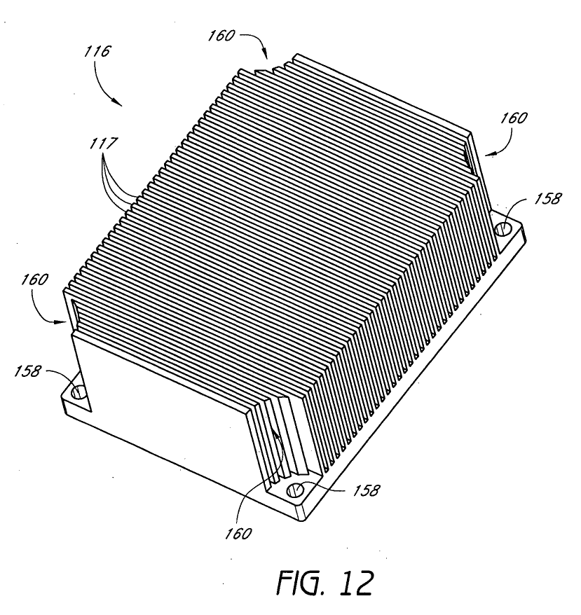

FIG. 12 shows heat sink 116. Holes 158 can be used for screws that attach heat sink 116 to other components of the system 110. Notches 160 allow a screw driver or other implements to access holes 158. FIG. 12 illustrates notches 160 formed in heat sink 116 by plates 117 that are progressively shorter near the ends of heat sink 116.

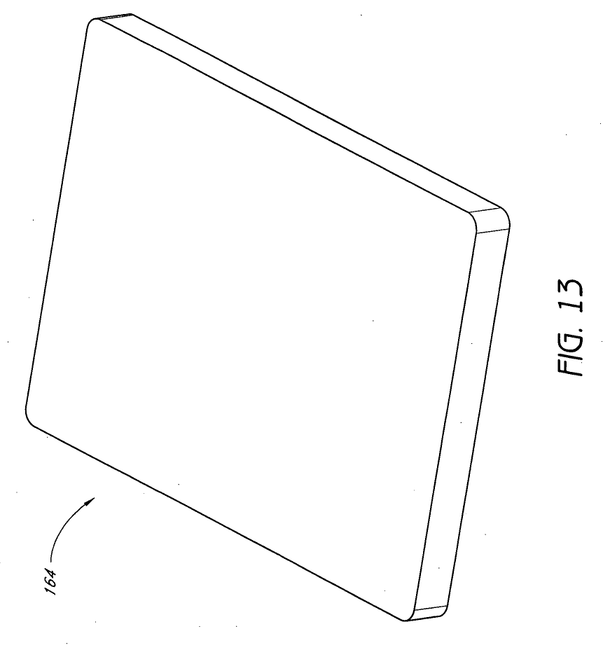

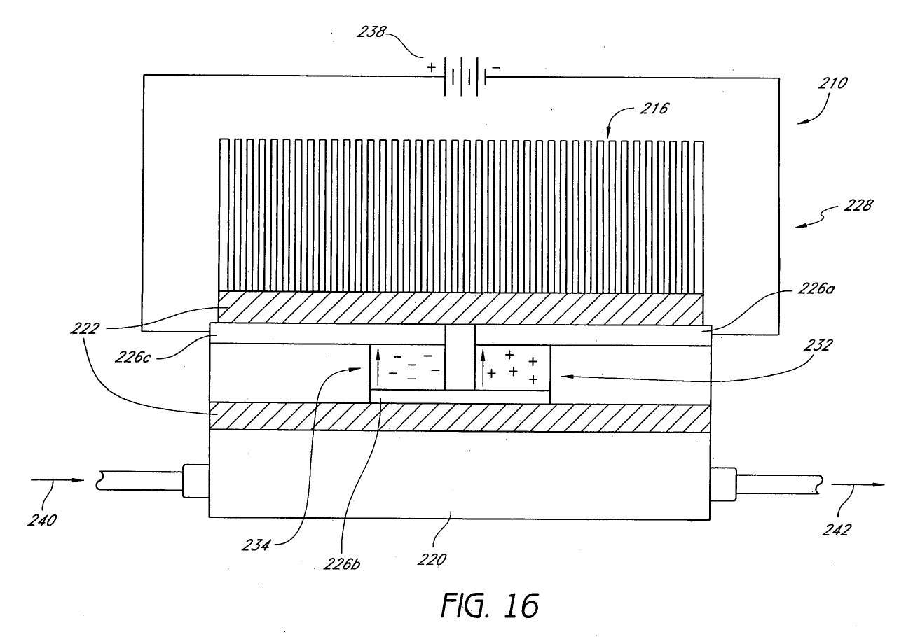

FIG. 13 shows a stylized representation of a Peltier module 164, which can function as a Peltier cooler. The function of a Peltier cooler is described below with reference to FIG. 16. The Peltier module 164 can include semiconductors, thermally conductive but electrically insulating materials, electrically conductive materials, hot junctions, cold junctions, etc.

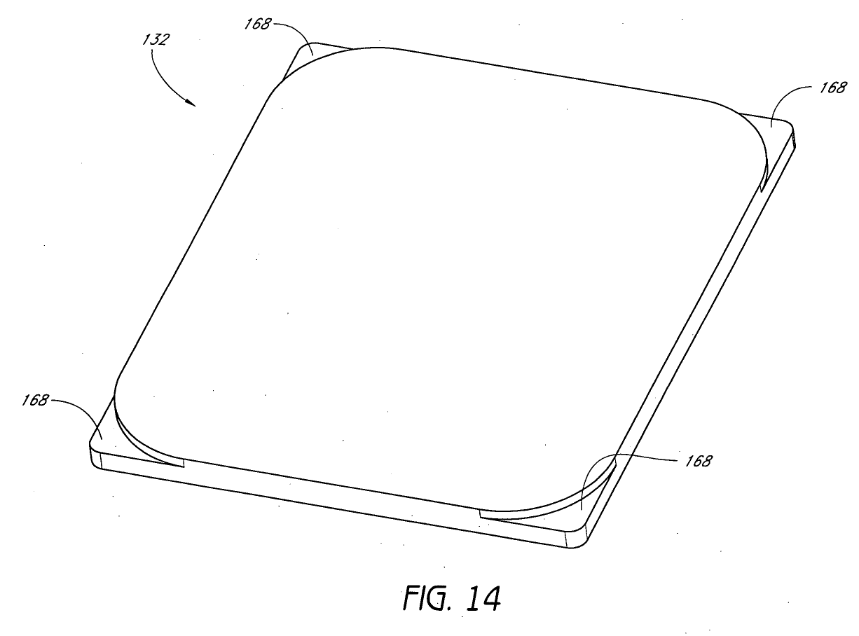

FIG. 14 shows an embodiment of a cold plate 132. The cold plate 132 has cutaway portions 168 at each of the four corners of the cold plate 132. The cold plate 132 is illustrated in FIG. 14 as seen from the bottom. The surface that is most visible in FIG. 14 is the same surface visible in FIG. 4. Cutaway portions 168 allow for the cold plate 132 to be retained within electrical assembly 134 while still exposing a large part of the surface of cold plate 132.

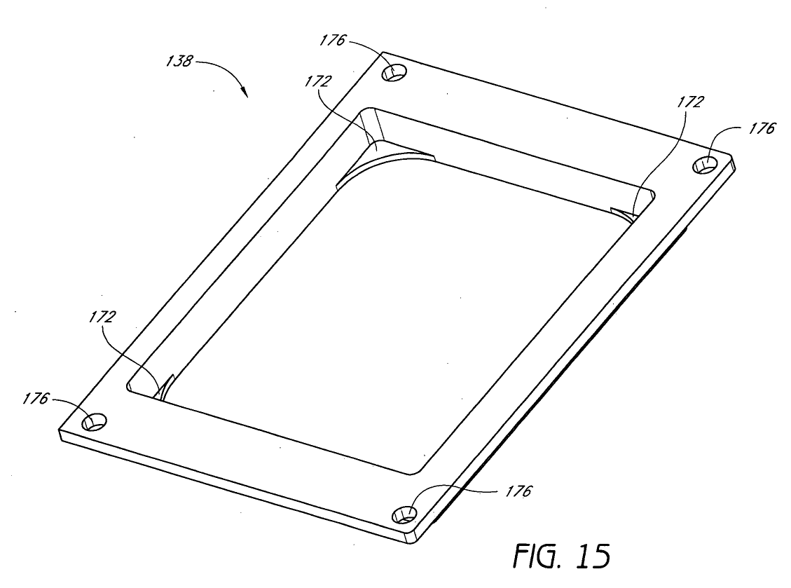

FIG. 15 illustrates bracket 138. Bracket 138 has four retaining portions 172 that fit into cutaway portions 168 of cold plate 132. Cold plate 132 can be positioned within bracket 138 with a thermoelectric device such as Peltier module 164 positioned immediately above cold plate 132. Both the cold plate 132 and the Peltier module 164 can be configured to be positioned within bracket 138. Bracket 138 can in turn be fastened to electrical assembly 134 and heat sink 116 using screws, for example. The screws can extend through holes 176.

FIG. 16 illustrates how a thermoelectric system 210 can function. A direct current source 238 supplies power to the system 210. Such power can be supplied by a vehicle battery or a portable battery, for example. Electrons flow through conductive wire 228 and through electrical conductors 226. A heat source 220 is cooled by the system 210. Heat is dispelled from the system 210 through heat sink 216. Electrical insulators 222 are also good heat conductors. The illustrated system 210 comprises a P-type semiconductor 232 and an N-type semi-conductor 234. Electrons flow from power supply 238 through conductive wire 228 into electrical conductor 226A. Electrons then pass through a hot junction into P-type semiconductor 232. As the electrons pass through this junction, they emit energy by moving into a lower energy level. The electrons then pass from the P-type semiconductor 232 through another conductive plate 226 into an N-type semiconductor 234. As the electrons move from the P-type semiconductor into the N-type semiconductor, they absorb energy as they move to a higher energy level. Thus, the junction between the P-type semiconductor and the N-type semiconductor through electrical conductor 226B is called the “cold junction.” As electrons continue from N-type semiconductor into electrical conductor 226C, the electrons release energy in the form of heat, which is then absorbed through electrical insulator 222 and dissipated to ambient air through heat sink 216. The electrons then move toward a positive end of power supply 238.

Heat source 220 can contain a fluid to be cooled. The fluid can flow into heat source 220 in a direction labeled by arrow 240. As the fluid flows in, it has a higher temperature. The fluid can exit heat source 220 in a direction shown by arrow 242. As the fluid exits heat source 220, the fluid has been cooled as a result of thermal conduction through the system 210. One example of a fluid to be cooled is blood drawn from a patient. For example, blood can be drawn from a carotid artery of a patient just before the blood enters the patient's brain. The blood can flow through heat source 220, where it is cooled by the system 210. When the fluid exits the system 210, it is cooler and can be returned to a carotid artery of the patient. The fluid can then flow into the brain through the natural arteries and veins, thus penetrating the brain and cooling the brain tissue in a generally even, efficient manner.

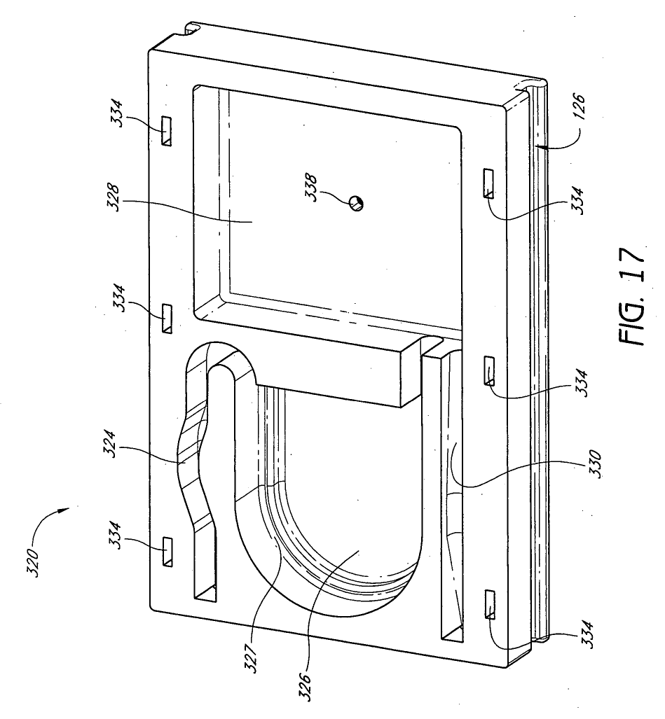

FIG. 17 shows the fluid assembly housing 320. The fluid assembly housing 320 is part of the fluid assembly 122 illustrated in FIGS. 2 and 3. The fluid assembly housing 320 comprises an inlet channel 324, a pump housing chamber 326, a coil housing chamber 328, and an outlet channel 330. The fluid assembly housing 320 can have six snap holes 334 in its upper surface, and a hole 338 in the coil housing chamber 328. The snap holes 334 can provide openings for snap protrusions (not shown) in electrical assembly 134 that can help the electrical assembly 134 and fluid assembly 122 mate together. A side channel 327 extends around the sidewall of pump housing chamber 326. As shown in FIGS. 2 and 3, a groove 126 extends along the length of fluid assembly housing 320. Fluid assembly housing 320 can comprise a molded plastic material. Such a material can provide for the economical manufacture of the fluid assembly housing 320. Furthermore, the fluid assembly housing 320 can be provided to a user in a sterile package, which can include appropriately-sized sterile tubing. Thus, some components of the disclosed device can be disposable, and provided in convenient form for use by medical professionals, medics on the battlefield, etc. Furthermore, plastic can provide advantageous thermally and electrically insulating properties. In one embodiment, the fluid assembly housing 320 is constructed from metal. This embodiment provides the advantage of being rugged and long-lasting. A metal, reusable fluid assembly housing 320 can house a sterile tube, or it can be easily sterilized using boiling water or other means.

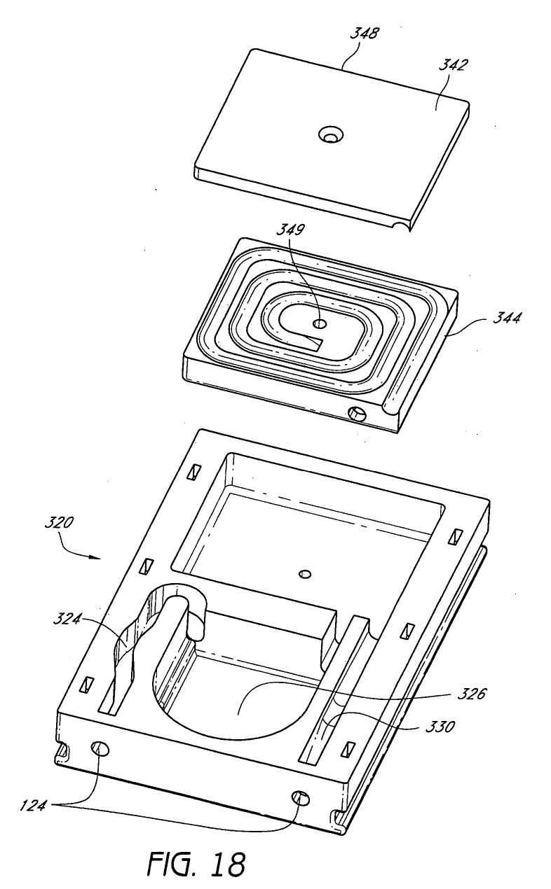

FIG. 18 illustrates how fluid assembly housing 320 fits together with top coil plate 342 and bottom coil plate 344. Bottom coil plate 344 can fit snugly within coil housing chamber 328, and top coil plate 342 cam fit on top of bottom coil plate 344, also within coil housing chamber 328. Top coil plate 342 has a hole 348 and bottom coil plate 344 has a hole 349. These holes can be aligned when the two plates are within coil housing chamber 328. Holes 348 and 349 can be configured to receive a screw that can fasten the two plates together and fasten them to the fluid assembly housing 320. FIG. 18 also illustrates holes 124 that lead to inlet channel 324 and outlet channel 330.

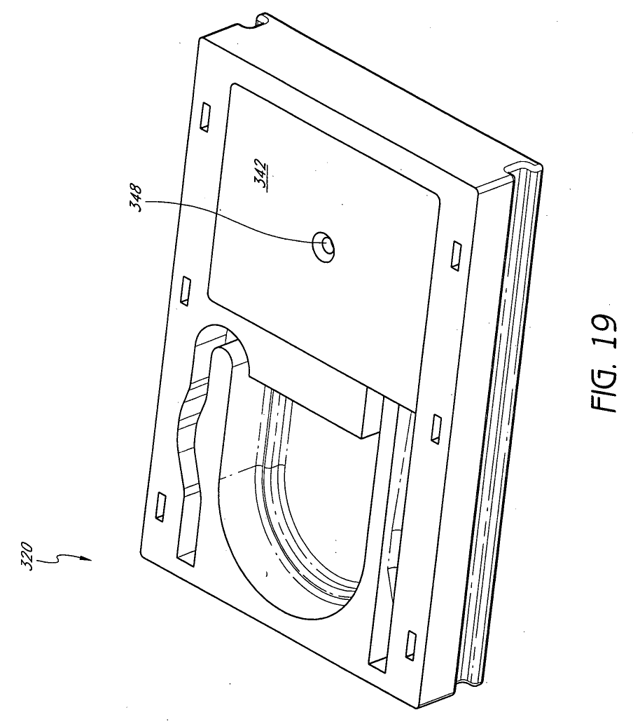

FIG. 19 shows fluid assembly housing 320 with top coil plate 342 visible within coil housing chamber 328. Hole 348 is also visible. The top, generally flat surface of top coil plate 324 can be formed from a heat-conductive metal, such as copper, aluminum, steel, alloys, etc. One advantageous configuration is flat, providing a convenient mating surface for the top coil plate 324 and cold plate 132. Heat can be transferred easily between these two flush surfaces when the fluid assembly 122 and electrical assembly 134 are mated together and aligned appropriately. One way to improve thermal contact between the two metal surfaces is to smear a thermally conductive substance such as a paste, cream, or gel on the two metal surfaces. For example, such a substance can advantageously be spread on the surface of top coil plate 342, when the fluid assembly 122 is manufactured and packaged for delivery. The paste can be overlaid by a removable film or layer. The removable layer can be peeled away by the user just before a disposable fluid assembly 122 and electrical assembly 134 are mated together for use, for example.

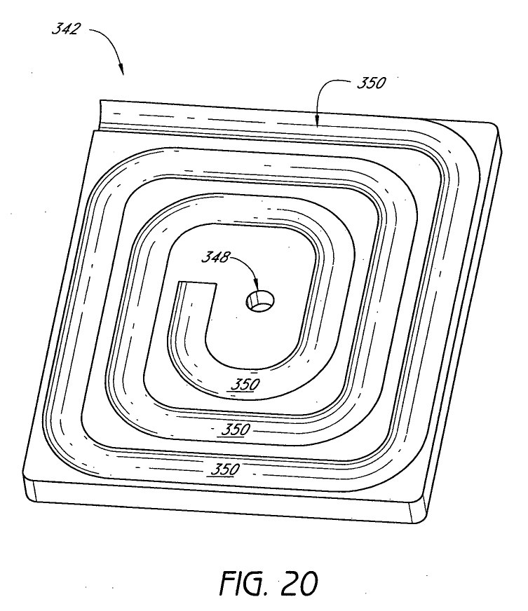

FIG. 20 illustrates top coil plate 342. Top coil plate 342 is seen from a different perspective in this figure. The figure illustrates that hole 348 extends completely through top coil plate 342. The bottom surface of top coil plate 342 has a coil channel 350 in its surface.

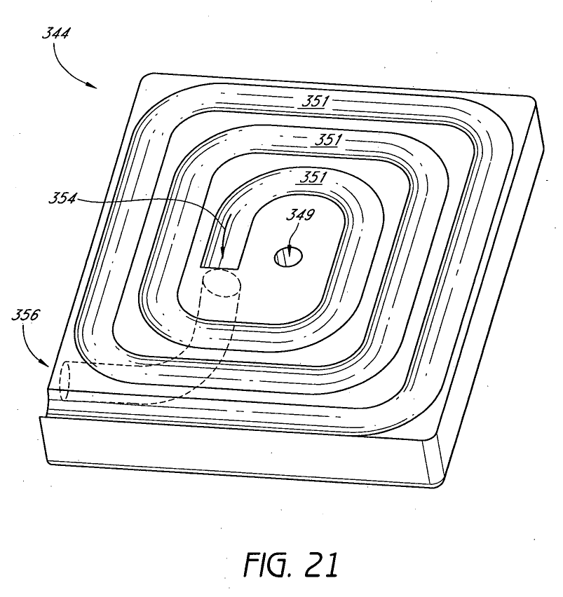

FIG. 21 illustrates another view of bottom coil plate 344. A bottom coil channel 351 extends in a coiled pattern in the top surface of bottom coil plate. When top coil plate 342 and bottom coil plate 344 are placed together with coil channel 350 and bottom coil channel 351 facing each other, the two channels 350 and 351 interface to form a tubular channel extending in a coil pattern between the two plates. Arrow 354 illustrates where the channel penetrates solely within bottom coil plate 344. Whereas for most of the coiled portion, the tubular channel is nested partially within the top coil plate 342 and partially within the bottom coil plate 344, the tubular channel is completely within bottom coil plate 344 from the point indicated by arrow 354 until the tubular channel opens on the side 356 of bottom coil plate 344. Because the completely enclosed portion of the tubular channel would not otherwise be visible in FIG. 21, it has been indicated with dashed lines where it extends beneath the surface of bottom coil plate 344. The tubular channel is configured to receive coiled, flexible tubing, through which fluid can flow. One example of a tubing that can be used is VYGON® tubing.

Top coil plate 342 and bottom coil plate 344 can be formed from any heat-conducting material. One advantageous material is metal. In a preferred embodiment, plates 342 and 344 are formed from copper or aluminum, where channels 350 and 351 have been milled using conventional machining techniques. Heat-conductive material provides the advantage of allowing heat energy to transfer from the fluid within the flexible tube, through the upper plate 342, and to be absorbed by the Peltier module 164.

In some embodiments, a thickness of top coil plate 342 is less than approximately 5 inches. In some embodiments, the thickness can be less than 2 inches. In other embodiments, the thickness can be less than 1 inch. In a preferred embodiment, a thickness of the top coil plate 342 is approximately 0.35 inches. The thickness can be chosen to allow appropriate heat transfer between the blood inside the coiled tube and a cooling device adjacent to top coil plate 342.

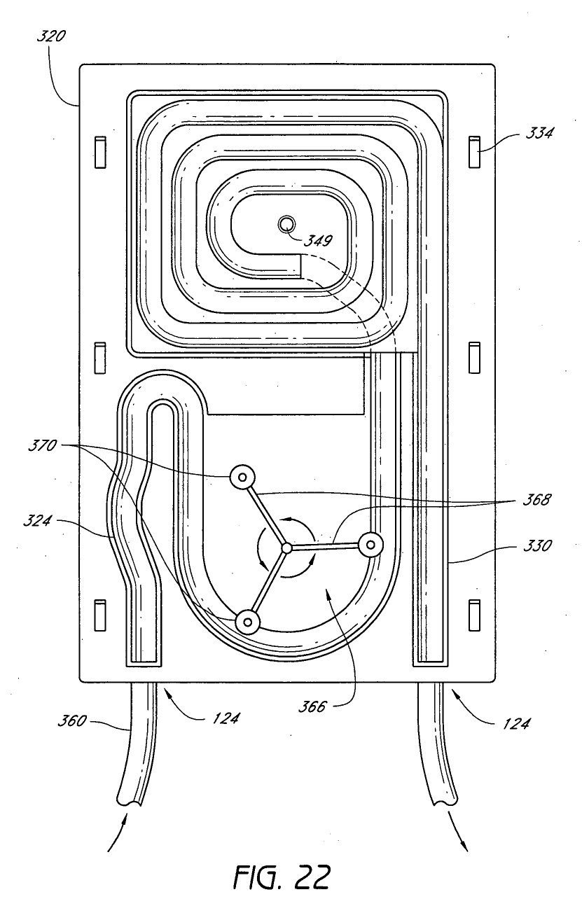

FIG. 22 illustrates how the operational elements fit into fluid assembly housing 320. A tube 360 enters the fluid assembly housing 320 through hole 124 and extends through inlet channel 324. Tube 360 further extends into pump housing chamber 326 and extends around the perimeter of the chamber 326 seated in side channel 327, illustrated in FIG. 17. The tube 360 then enters the side of bottom coil plate 344 on side 356 as illustrated in FIG. 21. The tube extends through the dotted channel and protrudes into bottom coil channel 351 near hole 349. The tube then coils around in ever larger loops until it extends into outlet channel 330 and through hole 124. In this way, tube 360 forms a continuous fluid pathway through fluid assembly housing 320.

Peristaltic pump 366 can be used to urge fluid to flow through tube 360. As illustrated, peristaltic pump 366 can have three arms 368, each having a roller 370. As the peristaltic pump 366 turns, as indicated by the arrows, the rollers 370 contact the tube 360. As the rollers 370 depress the sidewalls of the tube 360 and roll along the tube, fluid contained within the tube is urged to flow in a direction complimentary to the movement of the rollers 370. The rollers 370 can partially or completely compress the tube, depending on the tube's thickness, the length of arms 368, etc. Movement of fluid through the tube located within pump housing chamber 326 in turn causes fluid to flow throughout the length of the tube 360. Because the fluid within tube 360 is contained within a continuous fluid system, movement of fluid in one part of the tube 360 causes movement of fluid throughout the entire length of the tube 360. Peristaltic pump is driven by motor spindle 130, which extends from the motor 142 into the pump housing chamber 326.

A method of using an embodiment of the system described above can comprise determining the need to change the temperature of a region of a patient's body. A site for withdrawing and reintroducing blood can be chosen corresponding to part of the anatomy targeted for temperature adjustment. A fluid assembly can then be prepared for use by removing sterile packaging and threading a fluid tube through bottom coil plate 344. The fluid tube can then be coiled and laid into bottom coil channel 351. Top coil plate 342 can then be placed over the coiled tube such that the tube fits into both coil channel 350 and coil channel 351. The two ends of the fluid tube that extend out of the combined coil plates can then be arranged in the channels of fluid assembly housing 320. One end of the tube can be placed in outlet channel 330, and the other end is placed in side channel 327 and inlet channel 324. The two ends of the tube can then be threaded through holes 124 and attached to needles or some other structure such as a catheter for allowing fluid flow between a tube and a blood vessel of a patient.

The tube can be initially filled using blood from a non-target region of the patient's body. For example, a needle can be inserted into a femoral artery of the patient. This can help avoid inappropriate blood removal from the potentially more sensitive target region of the body. For example, many medical dangers can accompany a sudden draining of blood from the brain. Such dangers include electrolyte depletion, clot development, embolisms, plaque deposits, etc. Furthermore, a rapid change in blood pressure can cause a brain to be compressed into a brain stem, with harmful consequences. Thus, it can be advantageous to fill the tube prior to drawing blood from the target region. However, in some settings it may be preferable to drain bodily fluid from a region quickly in addition to cooling the temperature of the fluid. Under these circumstances, it may be desirable to allow the tube to fill initially with fluid from the target region.

After the two ends of the tube have been attached to needles and the target region chosen, the needles are inserted into the veins or arteries of the patient so that blood from the patient flows through the tube through the inlet channel 324 and into the fluid assembly 122. In order to encourage such flow, electrical assembly 134 can be mated to fluid assembly 122 and motor spindle 130 engages peristaltic pump 366. After a packaging film or layer (not shown) has been removed to uncover thermally conductive paste on the surface of top coil plate 342 (not shown), the mating of fluid assembly 122 and electrical assembly 134 brings cold plate 132 and top coil plate 342 into apposition. The metal surfaces of the two plates are flush—separated, if at all, only by a layer of thermally conductive paste. In practical effect, the thermally conductive paste enhances the thermal contact and transfer of energy between the two surfaces.

The electrical assembly 134 can be activated before or after blood has begun to flow through the fluid assembly 122 from the target area of the patient. After electrical assembly 134 and fluid assembly 122 have been mated together and the electrical assembly 134 has been activated, motor 142 turns peristaltic pump 366, driving rollers 370, which compress the tube as they roll around the perimeter of the pump housing chamber 326, urging the blood within the tube to flow through the tube. The blood continues to flow through the coiled region, where the blood temperature either increases or decreases, depending on the mode of operation of the Peltier module 164. If the electrical current running through Peltier module 164 flows in one direction, the cold junction or junctions are on the side closest to the fluid assembly 122. In this mode, blood and heat energy is drawn from the blood and dissipated from the heat sink 216 into the ambient air. If the electrical current running through Peltier module 164 flows in the other direction, the hot junction or junctions are on the side of the top coil plate 342 and energy flows into the blood, heating it.

After the fluid is cooled or heated, it continues to flow through the tube and reenters the target region of the patient's anatomy. Advantageously, the fluid path followed by the fluid is short after the blood has been heated or cooled, to minimize unwanted heating or cooling of the fluid and other inefficiencies.

Once the system 110 is functioning properly, the system 110 can be allowed to operate continuously to keep the blood at a desired temperature in the target region of the patient's body. In certain embodiments, the system 110 is portable and can be fastened to the patient or the patient's stretcher or clothes and transported with the patient. In certain embodiments, the system 110 draws low current and/or voltage and can be powered using a portable or vehicle battery. Advantageously, the system 110 can be unplugged from a control unit and maintain continuous operation during transportation of a patient.

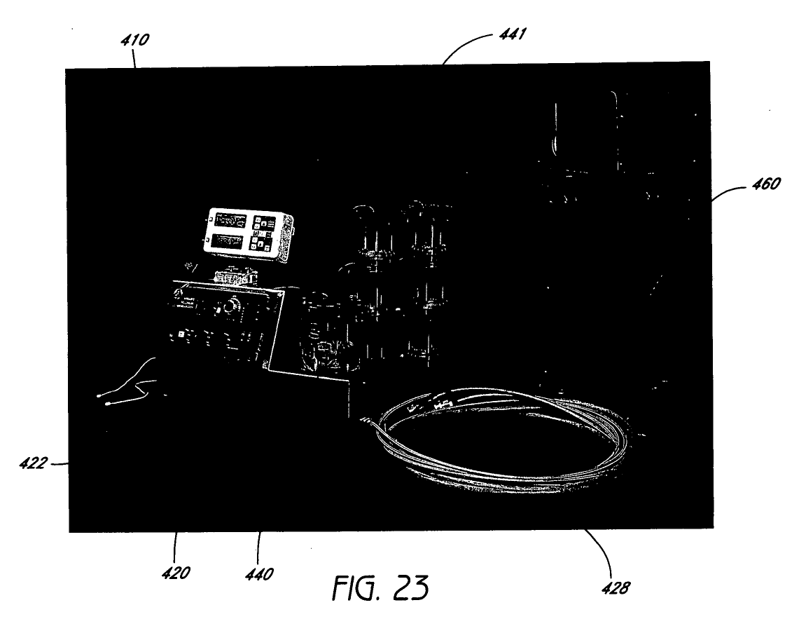

FIG. 23 shows an embodiment of a system 410 for controlling the temperature of a fluid. One example of such a fluid is blood. Other non-limiting examples include interstitial fluid, amniotic fluid, blood constituents, and lymphatic fluid. The figure illustrates a control module 420, a heat exchanger 440, a temperature module 460, tubing 428, temperature leads 422, and spare heat exchangers 441. Temperature leads 422 can be used to measure the temperature of fluid flowing to and from the subject. One possible subject is a rat; another possible subject is a human. The control module 420 can be used to choose various parameters, such as temperature and flow rate. Heat exchanger 440 provides an interface between a cooling or heating fluid and the fluid drawn from the subject. In one embodiment, the fluids are in thermal contact, but not direct contact. Tubing 428 can be used to connect the heat exchanger 440 to the temperature module 460. Temperature module 460 can generate a heated or cooled control fluid. The temperature module 460 can provide continuous control fluid flow through tubing 428.

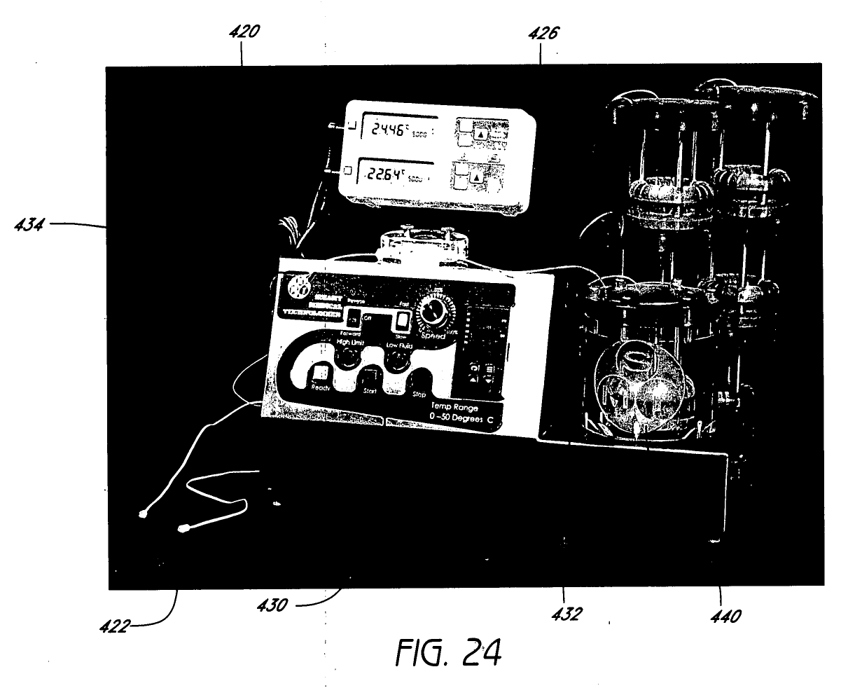

FIG. 24 shows a different view of control module 420. A peristaltic pump 426 is mounted on top of the control module 420. Fluid tube 432 runs through the peristaltic pump 426. At one end of fluid tube 432 is fluid lead 434. Fluid lead 434 can be connected through a needle to an artery or vein of the subject—for example, a human or other mammal. A rat can also be used as a test subject. Temperature leads 422 can be placed in thermal contact with the fluid flowing from the subject and into the control module or into thermal communication with the fluid flowing from the heat exchanger 440 toward the subject, or both. FIG. 24 shows a closer view of control panel 430.

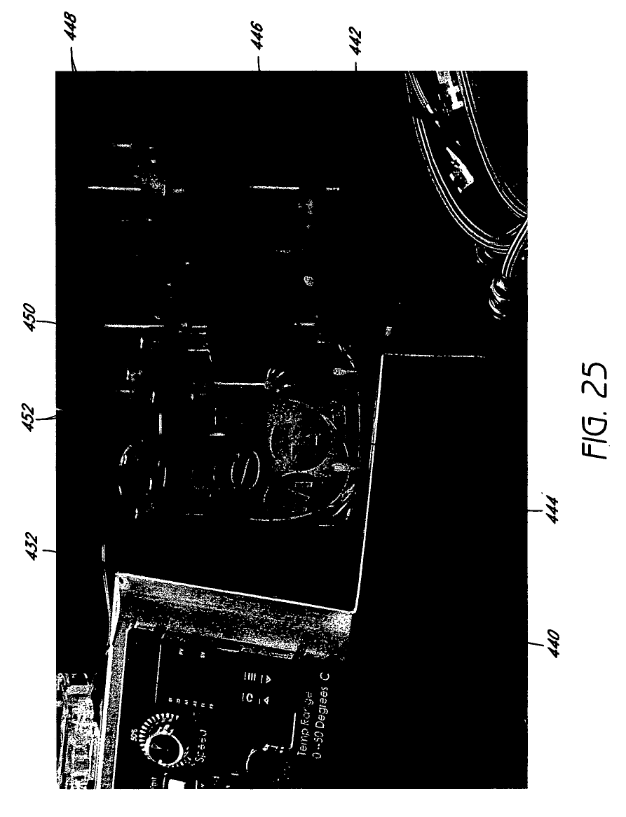

FIG. 25 illustrates a closer view of heat exchanger 440. Heat exchanger 440 comprises control fluid ports 442, control fluid holder 444, dipping torus 446, dipping rods 448, lid 450, and screws 452. Fluid from the subject, for example, blood, flows through fluid tube 430 after passing through the peristaltic pump 426. Fluid tube 432 extends through lid 450 and down into control fluid holder 444. Fluid tube 432 wraps around the upper and lower halves of dipping torus 446. Dipping torus 446 is held suspended inside control fluid holder 444 by dipping rods 448. After fluid tube 432 wraps around dipping torus 446, it extends up through lid 450 and continues to the subject, for example, a human. Tests can also be performed on a rat or a monkey, for example. Control fluid flows from temperature module 460 through tubing 428 into one of the control fluid ports 442. The control fluid ports are in fluid communication with control fluid holder 444. Control fluid can also flow out of one of control fluid ports 442 and back to temperature module 460. The temperature within control fluid holder 444 is thus controlled using control fluid. As this figure illustrates, two separate fluid systems interact at the heat exchanger 440, while retaining fluid independence. For example, blood may flow through fluid tube 432 and through multiple loops around dipping torus 446, while never physically mixing with control fluid within control fluid holder 444. However, the temperature of the blood within fluid tube 432 is affected by the temperature of control fluid, such as water, within the heat exchanger 440. The dipping torus 446 can be expanded as the top portion is raised up dipping rods 448 and farther away from the bottom portion of dipping torus 446. This forces the loops of fluid tube 432 to become longer to extend around the two halves of dipping torus 446. This, in turn, causes a greater length of fluid tube 432 to be in thermal contact with the control fluid bath in control fluid holder 444.

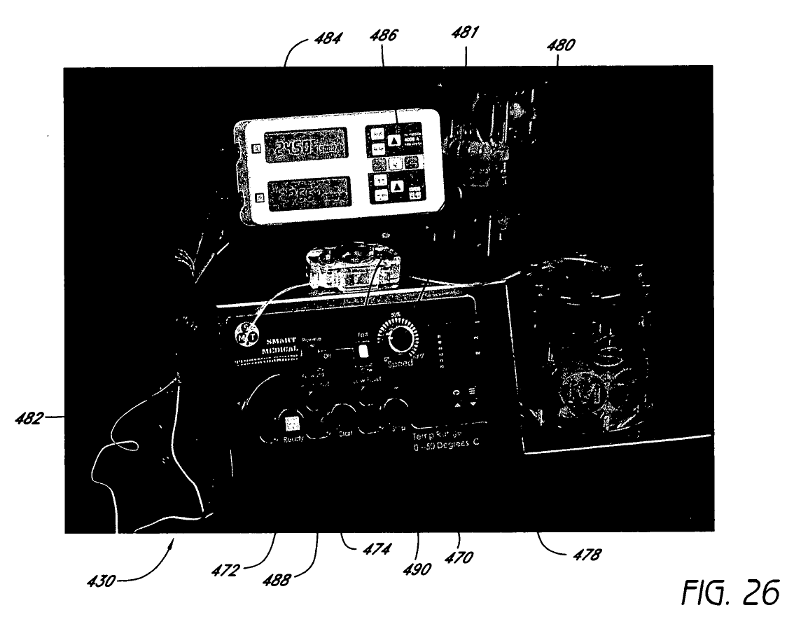

FIG. 26 shows a close-up view of control panel 430. Ready indicator 472 can be used to indicate the status of the system. Start button 474 can be used to activate the system. Stop button 476 can be used to deactivate the system. Temperature control 478 can be used to control the temperature of the control fluid. Low fluid light 490 can be used to warn when the fluid is low. High limit light 488 can be used to warn when the fluid is high. Pump switch 482 can be used to cause the pump to move fluid forward or in a reverse direction or turn off. Pump speed switch 481 can be used to cause the pump to move in fast mode or slow mode. Pump speed dial 480 can be used to control the speed of the pump in a more systematic way. Temperature screens 484 can be used to indicate the readings from thermistors associated with the fluid temperature going out of the subject and returning to the subject. Control panel 486 can be used to control the units and format of data readings and other readings shown on screens 484.

The system 410 illustrated in FIGS. 23 through 26 can operate in a similar way to the system 110. In one embodiment, however, the mechanism for heating or cooling fluid is different from the Peltier module 164. Instead, heat exchanger 440 provides an apparatus for heat exchange between two fluids. The system 410 also has many advantages relating to controllable parameters and data collection. In one embodiment, the system 410 can be used to perform multiple experiments to determine optimum conditions for cooling bodily fluids. Many variables can be adjusted independently using system 410. For example, flow rate can be adjusted through adjusting the speed and direction of the peristaltic pump 426. Additionally, the volume and temperature of fluid in the heat exchanger 440 can be independently adjusted, and each can even be varied over time and data collection can continue as each is adjusted. Furthermore, thermocouples can be used to collect data regarding the fluid temperature at various points in the system. In one embodiment, at least two different temperature readings can be obtained using temperature leads 422. In certain embodiments, the system 410 can comprise or interface with a computer. The computer (not shown) can control various components or aid in data collection and analysis, for example.

In some embodiments, a system can be adapted to integrate with a cervical collar or brace. For example, the embodiment of FIG. 2 can be fastened to a neck brace used for injured patients when there is a risk of spine injury. Such a configuration can include access holes for connecting needles and tubes, and accessing a carotid artery, for example. A neck brace can be advantageously combined with a fluid cooler so that a patient's head is stabilized and avoids undesired wounds from the access needles, for example.

In some embodiments, a system can be adapted to allow revascularization of a severed or partially severed limb or other anatomical structure. The tubing and connections can be configured to allow blood to continue to flow through tissue, revitalizing or preserving the tissue's viability. Furthermore, a fluid temperature control system can be used to cool or heat severed tissue to slow necrosis, slow or hasten metabolic processes, etc.

The foregoing description provides examples of certain embodiments of the inventions. Many variations in the disclosed structure and features will be apparent to those skilled in the art after reading this disclosure, and such variations are within the scope of the inventions in this application.

What is claimed is:

1. An apparatus for affecting the temperature of a bodily organ comprising:
a fluid-drawing device;
an extra-bodily fluid pathway;
a temperature-affecting device in at least partial contact with the extra-bodily fluid pathway;
a pump; and
a fluid-insertion device.

2. The apparatus of claim 1, wherein the fluid-drawing device and the fluid-insertion device are both needles.

3. The apparatus of claim 1, wherein the temperature-affecting device is a thermoelectric device.

4. The apparatus of claim 1, wherein the temperature-affecting device is a Peltier device.

5. The apparatus of claim 1, wherein the temperature-affecting device is a resistive heater.

6. The apparatus of claim 1, wherein the temperature-affecting device comprises a heat sink.

7. The apparatus of claim 1, wherein the extra-bodily fluid pathway comprises medical tubing.

8. The apparatus of claim 1, wherein the extra-bodily fluid pathway comprises sterilized tubing.

9. The apparatus of claim 1, wherein at least a portion of the extra-bodily fluid pathway is disposable.

10. The apparatus of claim 1, wherein the extra-bodily fluid pathway is configured to increase thermal contact between the extra-bodily fluid pathway and the temperature-changing device.

11. The apparatus of claim 10, wherein the extra-bodily fluid pathway comprises a tube that is curved along a region of thermal contact with a portion of the temperature-changing device.

12. The apparatus of claim 1, wherein the extra-bodily fluid pathway further comprises a first outflow portion configured to contain fluid flowing from a first living entity and a second inflow portion configured to contain fluid flowing to a second living entity.

13. The apparatus of claim 12, wherein the first and second living entities are the same human being.

14. The apparatus of claim 12, wherein the first and second living entities are the same living organ.

15. The apparatus of claim 12, wherein the inflow portion is shorter than the outflow portion.

16. The system of claim 1, wherein the pump is a peristaltic pump.

17. The apparatus of claim 1, further comprising a pump motor.

18. The apparatus of claim 17, further comprising a control unit configured to interface with the pump motor and/or the temperature-affecting device.

19. The apparatus of claim 18, wherein the control unit is configured to control a temperature of the temperature-affecting device and/or control a pump speed.

20-64. (canceled)
